(19) United States
(12) Patent Application Publication
(10) Pub. No.: US 2006/0247257 A1
(43) Pub. Date: Nov. 2, 2006

(54) Combination

(76) Inventor: John Dixon, Leicestershire (GB)

Correspondence Address: FISH & RICHARDSON P.C., P.O BOX 1022, MINNEAPOLIS, MN, 55440-1022, US

(21) Appl. No.: 10/545,972

(22) PCT Filed: Feb. 16, 2004
(86) PCT No.: PCT/SE04/00196

§ 371 Date: Aug. 17, 2005

(30) Foreign Application Priority Data
Feb. 18, 2003 (SE) 0300445-4

(51) Int. Cl. A61K 31/495; A61K 31/4412; A61K 31/44; A61K 31/4709; A61K 31/4172; A61K 31/165

(52) U.S. Cl. 514/255.01; 514/355; 514/352; 514/602; 514/617; 514/400; 514/314

(57) ABSTRACT

The invention provides a pharmaceutical composition, pharmaceutical product or kit comprising a first active ingredient which is a P2X₇ receptor antagonist, and a second active ingredient which is an inhibitor of proTNFα; convertase enzyme (TACE), for use in the treatment of inflammatory disorders.

The present invention relates to combinations of pharmaceutically active substances for use in the treatment of inflammatory conditions/disorders, especially rheumatoid arthritis.

Chronic inflammatory disorders such as rheumatoid arthritis are polygenic, highly complex, and involve multiple inflammatory and immune mechanisms. Treatment of these disorders has been largely empirical with a variety of therapeutic agents being used with little understanding of the mechanisms involved. Recent research suggests that two inflammatory mediators, the cytokines IL-1 and TNFalpha (TNFα), may play key roles in the inflammatory process in rheumatoid arthritis.

It would be desirable to develop new pharmaceuticals for use in treating inflammatory conditions/disorders.

In accordance with the present invention, there is therefore provided a pharmaceutical composition comprising, in admixture, a first active ingredient which is a P2X₇ receptor antagonist, and a second active ingredient which is an inhibitor of proTNFα convertase enzyme (TACE).

The P2X₇ receptor (previously known as P2Z receptor) is a ligand-gated ion channel that is present on a variety of cell types, largely those known to be involved in the inflammatory/immune process, specifically, macrophages, mast cells and lymphocytes (T and B). Activation of the P2X₇ receptor by extracellular nucleotides, in particular adenosine triphosphate, is known to lead, amongst other things, to the release of interleukin-1β (IL-1β).

An antagonist of the P2X₇ receptor is a compound or other substance that is capable of preventing, whether fully or partially, activation of the P2X₇ receptor.

Methods for assaying for P2X₇ receptor antagonism are known in the art, for example from WO 01/42194 which describes an assay based on the observation that when the P2X₇ receptor is activated using a receptor agonist in the presence of ethidium bromide (a fluorescent DNA probe), an increase in the fluorescence of intracellular DNA-bound ethidium bromide is observed. Thus, an increase in fluorescence can be used as a measure of P2X₇ receptor activation and therefore to quantify the effect of a compound or substance on the P2X₇ receptor.

In WO 01/42194, the assay is carried out by taking a 96-well flat bottomed microtitre plate and filling the wells with 250 μl of test solution comprising 200 μl of a suspension of THP-1 cells (2.5×10⁶ cells/ml) containing 10 M ethidium bromide, 25 μl of a high potassium buffer solution containing 10⁻⁵ M benzoylbenzoyl adenosine triphosphate (bbATP, a known P2X₇ receptor agonist), and 25 μl of the high potassium buffer solution containing 3×10⁻⁵ M test compound. The plate is covered with a plastics sheet and incubated at 37° C. for one hour. The plate is then read in a Perkin-Elmer fluorescent plate reader, excitation 520 nm, emission 595 nm, slit widths: Ex 15 nm, Em 20 nm. For the purposes of comparison, bbATP (a P2X₇ receptor agonist) and pyridoxal 5-phosphate (a P2X₇ receptor antagonist) are used separately in the test as controls. From the readings obtained, a pIC₅₀ figure is calculated for the test compound, this figure being the negative logarithm of the concentration of test compound necessary to reduce the bbATP agonist activity by 50%. A pIC₅₀ figure greater than 5.5 is normally indicative of an antagonist.

TACE (also known as ADAM17) which has been isolated and cloned [R. A. Black et al. (1997) Nature 385:729-733; M. L. Moss et al. (1997) Nature 385:733-736] is a member of the admalysin family of metalloproteins. TACE has been shown to be responsible for the cleavage of pro-TNFα, a 26 kDa membrane bound protein to release 17 kDa biologically active soluble TNFα [Schlondorff et al. (2000) Biochem. J. 347: 131-138]. TACE mRNA is found in most tissues, however TNFα is produced primarily by activated monocytes, macrophages and T lymphocytes involved in the inflammatory/immune process.

An inhibitor of TACE is a compound or other substance that is capable of inhibiting the activity of proTNFα convertase enzyme, whether fully or partially.

The ability of a compound or substance to inhibit proTNFα convertase enzyme (TACE) may be assessed using a partially purified, isolated enzyme assay, the enzyme being obtained from the membranes of THP-1 as described by K. M. Mohler et al., (1994) Nature 370:218-220. The purified enzyme activity and inhibition thereof is determined by incubating the partially purified enzyme in the presence or absence of test compounds using the substrate 4′,5′-Dimethoxy-fluoresceinyl Ser.Pro.Leu.Ala.Gln.Ala.Val.-Arg.Ser.Ser.Ser.Arg.Cys(4-(3-succinimid-1-yl)-fluorescein)-NH₂ in assay buffer (50 mM Tris HCl, pH 7.4 containing 0.1% (w/v) Triton X-100 and 2 mM CaCl₂), at 26° C. for 4 hours. Activity is determined by measuring the fluorescence at λex 485 nm and λem 538 nm. Percent inhibition is calculated as follows: % Inhibition is equal to the [Fluorescence_{plus inhibitor}−Fluorescencebackground] divided by the [Fluorescence_{minus inhibitor}−Fluorescence_{background}].

The substrate may be synthesised as follows. The peptidic part of the substrate is assembled on Fmoc-NH-Rink-MBHA-polystyrene resin either manually or on an automated peptide synthesiser by standard methods involving the use of Fmoc-amino acids and O-benzotriazol-1-yl-N,N,N′,N′-tetramethyluronium hexafluorophosphate (HBTU) as coupling agent with at least a 4- or 5-fold excess of Fmoc-amino acid and HBTU. Ser¹ and Pro² are double-coupled. The following side chain protection strategy is employed; Ser¹(But), Gln⁵(Trityl), Arg^{8,12}(Pmc or Pbf), Ser^{9,10,11}(Trityl), Cys¹³(Trityl). Following assembly, the N-terminal Fmoc-protecting group is removed by treating the Fmoc-peptidyl-resin in dimethyl formamide (DMF). The amino-peptidyl-resin so obtained is acylated by treatment for 1.5-2 hr at 70° C. with 1.5-2 equivalents of 4′,5′-dimethoxy-fluorescein-4(5)-carboxylic acid [Khanna & Ullman, (1980) Anal Biochem. 108:156-161] which has been preactivated with diisopropylcarbodiimide and 1-hydroxybenzotriazole in DMF. The dimethoxyfluoresceinyl-peptide is then simultaneously deprotected and cleaved from the resin by treatment with trifluoroacetic acid containing 5% each of water and triethylsilane. The dimethoxyfluoresceinyl-peptide is isolated by evaporation, triturated with diethyl ether and filtered. The isolated peptide is reacted with 4-(N-maleimido)-fluorescein in DMF containing diisopropylethylamine, the product is purified by RP-HPLC and finally isolated by freeze-drying from aqueous acetic acid. The product can be characterised by MALDI-TOF MS and amino acid analysis.

Examples of P2X₇ receptor antagonists include the compounds described in WO 00/61569, WO 01/42194, WO 01/44170 and WO 03/041707, the entire contents of which are incorporated herein by reference.

More specifically, WO 00/61569 discloses a compound of formula 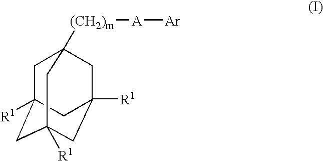
wherein m represents 1, 2 or 3;
each R¹ independently represents a hydrogen or halogen atom;
A represents C(O)NH or NHC(O);
Ar represents a group 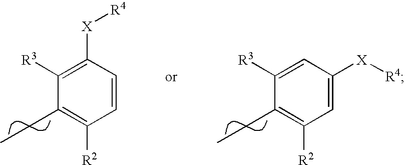
X represents a bond, an oxygen atom or a group CO, (CH₂)₁₋₆, CH═, (CH₂)₁₋₆O, O(CH₂)₁₋₆, O(CH₂)₂₋₆O, O(CH₂)₂₋₃O(CH₂)₁₋₃, CR′(OH), (CH₂)₁₋₃O(CH₂)₁₋₃, (CH₂)₁₋₃O(CH₂)₂₋₃O, NR⁵, (CH₂)₁₋₆NR⁵(CH₂)₁₋₆, (CH₂)₁₋₃NR⁵(CH₂)₁₋₃, O(CH₂)₂₋₆NR⁵, O(CH₂)₂₋₃NR⁵(CH₂)₁₋₃, (CH₂)₁₋₃NR⁵(CH₂)₂₋₃O, NR⁵(CH₂)₂₋₆O, NR⁵(CH₂)₂₋₃O(CH₂)₁₋₃, CONR⁵, NR⁵ CO, S(O)ₙ, S(O)ₙCH₂, CH₂S(O)ₙ, SO₂NR⁵ or NR⁵SO₂;
n is 0, 1 or 2;
R′ represents a hydrogen atom or a C₁-C₆ alkyl group;
one of R² and R³ represents a halogen, cyano, nitro, amino, hydroxyl, or a group selected from (i) C₁-C₆ alkyl optionally substituted by at least one C₃-C₆ cycloalkyl, (ii) C₃-C₈ cycloalkyl, (iii) C₁-C₆ alkyloxy optionally substituted by at least one C₃-C₆ cycloalkyl, and (iv) C₃-C₈ cycloalkyloxy, each of these groups being optionally substituted by one or more fluorine atoms, and the other of R² and R³ represents a hydrogen or halogen atom;
either R⁴ represents a 3- to 9-membered saturated or unsaturated aliphatic heterocyclic ring system containing one or two nitrogen atoms and optionally an oxygen atom, the heterocyclic ring system being optionally substituted by one or more substituents independently selected from fluorine atoms, hydroxyl, carboxyl, cyano, C₁-C₆ alkyl, C₁-C₆ hydroxyalkyl, —NR⁶R⁷, —(CH₂)ᵣNR⁶R⁷ and —CONR⁶R⁷,
or R⁴ represents a 3- to 8-membered saturated carbocyclic ring system substituted by one or more substituents independently selected from —NR⁶R⁷, —(CH₂)ᵣNR⁶R⁷ and —CONR⁶R⁷, the ring system being optionally further substituted by one or more substituents independently selected from fluorine atoms, hydroxyl and C₁-C₆ alkyl;
r is 1, 2, 3, 4, 5 or 6;
R⁵ represents a hydrogen atom or a C₁-C₆ alkyl or C₃-C₈ cycloalkyl group;
R⁶ and R⁷ each independently represent a hydrogen atom or a C₁-C₆ alkyl, C₂-C₆ hydroxyalkyl or C₃-C₈ cycloalkyl group, or R⁶ and R⁷ together with the nitrogen atom to which they are attached form a 3- to 8-membered saturated heterocyclic ring; with the provisos that,
(a) when A represents C(O)NH and R⁴ represents an unsubstituted 3- to 8-membered saturated aliphatic heterocyclic ring system containing one nitrogen atom, then X is other than a bond, and
(b) when A represents C(O)NH and X represents a group (CH₂)₁₋₆ or O(CH₂)₁₋₆, then R⁴ does not represent an unsubstituted imidazolyl, unsubstituted morpholinyl, unsubstituted piperidinyl or unsubstituted pyrrolidinyl group, and
(c) when A represents NHC(O) and R⁴ represents an unsubstituted 3- to 8-membered saturated aliphatic heterocyclic ring system containing one nitrogen atom, then X is other than a bond, and
(d) when A represents NHC(O) and X represents O(CH₂)₁₋₆, NH(CH₂)₁₋₆ or SCH₂, then R⁴ does not represent an unsubstituted 1-piperidinyl or unsubstituted 1-pyrrolidinyl group, and
(e) when A represents NHC(O) and X represents O(CH₂)₂₋₃NH(CH₂)₂, then R⁴ does not represent an imidazolyl group;
or a pharmaceutically acceptable salt or solvate thereof

WO 01/42194 discloses a compound of formula 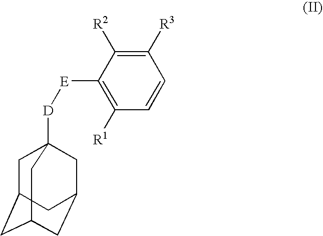
wherein D represents CH₂ or CH₂CH₂;
E represents C(O)NH or NHC(O);
R¹ and R² each independently represent a hydrogen or halogen atom, or an amino, nitro, C₁-C₆ alkyl or trifluoromethyl group;
R³ represents a group of formula 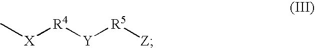
X represents an oxygen or sulphur atom or a group NH, SO or SO₂;
Y represents an oxygen or sulphur atom or a group NR¹¹, SO or SO₂;
Z represents a group —OH, —SH, —CO₂H, C₁-C₆ alkoxy, C₁-C₆ alkylthio, C₁-C₆-alkylsulphinyl, C₁-C₆-alkylsulphonyl, —NR⁶R⁷, —C(O)NR⁸R⁹, imidazolyl, 1-methylimidazolyl, —N(R¹⁰)C(O)—C₁-C₆ alkyl, C₁-C₆ alkylcarbonyloxy, C₁-C₆ alkoxycarbonyloxy, —OC(O)NR¹²R¹³, —OCH₂OC(O)R¹⁴, —OCH₂OC(O)OR¹⁵ or —OC(O)OCH₂OR¹⁶;
R⁴ represents a C₂-C₆ alkyl group;
R⁵ represents a C₁-C₆ alkyl group;
R⁶, R⁷, R⁸, R⁹, R¹⁰, R¹² and R¹³ each independently represent a hydrogen atom, or a C₁-C₆ alkyl group optionally substituted by at least one hydroxyl group;
R¹¹ represents a hydrogen atom, or a C₁-C₆ alkyl group optionally substituted by at least one substituent independently selected from hydroxyl and C₁-C₆ alkoxy; and
R¹⁴, R¹⁵ and R¹⁶ each independently represent a C₁-C₆ alkyl group; with the provisos that (i) when E represents NHC(O), X represents O, S or NH and Y represents O, then Z represents —NR⁶R⁷ where R⁶ represents a hydrogen atom and R⁷ represents either a hydrogen atom or a C₁-C₆ alkyl group substituted by at least one hydroxyl group, and (ii) when E represents NHC(O), X represents O, S or NH, Y represents NH and R⁵ represents CH₂CH₂, then Z is not —OH or imidazolyl;
or a pharmaceutically acceptable salt or solvate thereof.

WO 01/44170 discloses a compound of formula 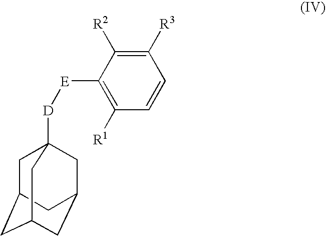
wherein D represents CH₂ or CH₂CH₂;
represents C(O)NH or NHC(O);
R¹ and R² each independently represent hydrogen, halogen, amino, nitro, C₁-C₆ alkyl or trifluoromethyl, but R¹ and R² may not both simultaneously represent hydrogen;
R³ represents a group of formula 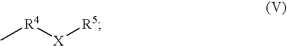
R⁴ represents a C₁-C₆ alkyl group;
X represents an oxygen or sulphur atom or a group NR¹³, SO or SO₂;
R⁵ represents hydrogen, or R⁵ represents C₁-C₆ alkyl or C₂-C₆ alkenyl, each of which may be optionally substituted by at least one substituent selected from halogen, hydroxyl, (di)-C₁-C₆-alkylamino, —Y—R⁶, 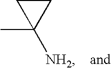
a 5- or 6-membered heteroaromatic ring comprising from 1 to 4 heteroatoms independently selected from nitrogen, oxygen and sulphur which heteroaromatic ring may itself be optionally substituted by at least one substituent selected from halogen, hydroxyl and C₁-C₆ alkyl;
Y represents an oxygen or sulphur atom or a group NH, SO or SO₂;
R⁶ represents a group —R⁷Z where R⁷ represents a C₂-C₆ alkyl group and Z represents an —OH, —CO₂H, —NR⁸R⁹, —C(O)NR¹⁰R¹¹ or —N(R¹²)C(O)—C₁-C₆ alkyl group, and, in the case where Y represents an oxygen or sulphur atom or a group NH, R⁶ additionally represents hydrogen, C₁-C₆ alkyl, C₁-C₆ alkylcarbonyl, C₁-C₆ alkoxycarbonyl, —C(O)NR¹⁴R¹⁵, —CH₂OC(O)R¹⁶, —CH₂OC(O)OR¹⁷ or —C(O)OCH₂OR¹⁸;
R⁸, R⁹, R¹⁰, R¹¹ and R¹² each independently represent a hydrogen atom or a C₁-C₆ alkyl group;
R¹³ represents hydrogen, C₃-C₈ cycloalkyl, C₃-C₈ cycloalkylmethyl, or R¹³ represents a C₁-C₆ alkyl group optionally substituted by at least one substituent selected from hydroxyl and C₁-C₆ alkoxy; and
R¹⁴, R¹⁵, R¹⁶, R¹⁷ and R¹⁸ each independently represent a C₁-C₆ alkyl group;
with the proviso that when E is C(O)NH, X is O, NH or N(C₁-C₆ alkyl), then R⁵ is other than a hydrogen atom or an unsubstituted C₁-C₆ alkyl group;
or a pharmaceutically acceptable salt or solvate thereof.

Preferred compounds of formula (IV) are those wherein R⁵ represents an optionally substituted C₁-C₆ alkyl group. A preferred substituent is —Y—R⁶.

When R⁵ is substituted with a 5- or 6-membered heteroaromatic ring comprising from 1 to 4 heteroatoms, it is preferred that the number of nitrogen atoms in the heteroaromatic ring is not greater than 2.

WO 03/041707 discloses a compound of formula 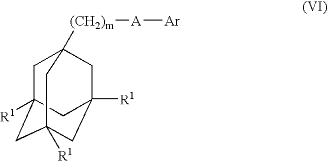
wherein m represents 1, 2 or 3;
each R¹ independently represents a hydrogen or halogen atom;
A represents C(O)NH or NHC(O);
Ar represents a group 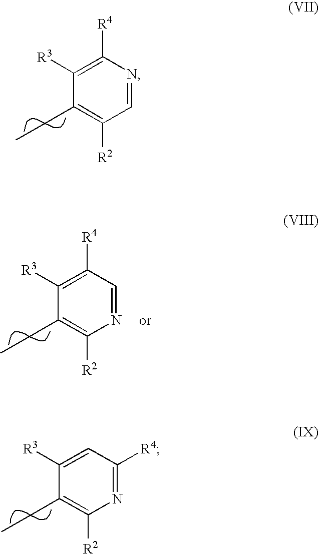
one of R² and R³ represents halogen, nitro, amino, hydroxyl, or a group selected from (i) C₁-C₆ alkyl optionally substituted by at least one halogen atom, (ii) C₃-C₈ cycloalkyl, (iii) C₁-C₆ alkoxy optionally substituted by at least one halogen atom, and (iv) C₃-C₈ cycloalkyloxy, and the other of R² and R³ represents a hydrogen or halogen atom;
R⁴ represents a group 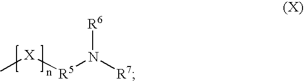
X represents an oxygen or sulphur atom or a group >N—R⁸;
n is 0 or 1;
R⁵ represents a C₁-C₅ alkyl group which may be optionally substituted by at least one substituent selected from hydroxyl, halogen and C₁-C₆ alkoxy;
R⁶ and R⁷ each independently represent a hydrogen atom, C₁-C₆ alkyl (optionally substituted by at least one substituent selected from hydroxyl, halogen, C₁-C₆ alkoxy, and (di)-C₁-C₄ alkylamino (itself optionally substituted by at least one hydroxyl group)), or C₃-C₈ cycloalkyl (optionally substituted by at least one substituent selected from hydroxyl, halogen and C₁-C₆ alkoxy); and
R⁸ represents a hydrogen atom or a C₁-C₅₀ group which may be optionally substituted by at least one substituent selected from hydroxyl, halogen and C₁-C₆ alkoxy;
with the provisos that:
(a) when n is 0, then A is NHC(O), and
(b) when n is 1, X represents oxygen and A is C(O)NH, then R⁶ and R⁷ do not both simultaneously represent a hydrogen atom or do not both simultaneously represent an unsubstituted C₁-C₆ alkyl, or when one of R⁶ and R⁷ represents a hydrogen atom, then the other of R⁶ and R⁷ does not represent an unsubstituted C₁-C₆ alkyl; and
(c) when n is 1, X is oxygen, sulphur or >NH and A is NHC(O), then R⁶ and R⁷ do not both simultaneously represent a hydrogen atom or do not both simultaneously represent an unsubstituted C₁-C₆ alkyl, or when one of R⁶ and R⁷ represents a hydrogen atom, then the other of R⁶ and R⁷ does not represent an unsubstituted C₁-C₆ alkyl or —CH₂CH₂OH;
   or a pharmaceutically acceptable salt or solvate thereof.

In an embodiment of the invention, the P2X₇ receptor antagonist is
2-Chloro-5-[[2-(2-hydroxy-ethylamino)-ethylamino]-methyl]-N-(tricyclo[3.3.1.1^{3,7}]dec-1-ylmethyl)-benzamide, dihydrochloride,
2-Chloro-5-[3-[(3-hydroxypropyl)amino]propyl]-N-(tricyclo[3.3.1.1]dec-1-ylmethyl)-benzamide,
(R)-2-Chloro-5-[3-[(2-hydroxy-1-methylethyl)amino]propyl]-N-(tricyclo[3.3.1.1^{3,7}]dec-1-ylmethyl)-benzamide,
2-Chloro-5-[[2-[(2-hydroxyethyl)amino]ethoxy]methyl]-N-(tricyclo[3.3.1.1^{3,7}]dec-1-ylmethyl)-benzamide,
2-Chloro-5-[3-[3-(methylamino)propoxy]propyl]-N-(tricyclo[3.3.1.1^{3,7}]dec-1-ylmethyl)benzamide,
2-Chloro-5-[3-(3-hydroxy-propylamino)-propoxy]-N-(tricyclo[3.3.1.1^{3,7}]dec-1-ylmethyl)-benzamide,
2-Chloro-5-[2-(3-hydroxypropylamino)ethylamino]-N-(tricyclo[3.3.1.1^{3,7}]dec-1-ylmethyl)-benzamide,
2-Chloro-5-[2-(3-hydroxypropylsulfonyl)ethoxy]-N-(tricyclo[3.3.1.1^{3,7}]dec-1-ylmethyl)-benzamide,
2-Chloro-5-[2-[2-[(2-hydroxyethyl)amino]ethoxy]ethoxy]-N-(tricyclo[3.3.1.1^{3,7}]dec-1-ylmethyl)-benzamide,
2-Chloro-5-[[2-[[2-(1-methyl-1H-imidazol-4-yl)ethyl]amino]ethyl]amino]-N-(tricyclo[3.3.1.1^{3,7}]dec-1-ylmethyl)-benzamide,
2-Chloro-5-piperazin-1-ylmethyl-N-(tricyclo[3.3.1.1]dec-1-ylmethyl)-benzamide,
2-Chloro-5-(4-piperidinyloxy)-N-(tricyclo[3.3.1.1^{3,7}]dec-1-ylmethyl)-benzamide,
2-Chloro-5-(2,5-diazabicyclo[2.2.1]hept-2-ylmethyl)-N-(tricyclo[3.3.1.1]dec-1-ylmethyl)-benzamide,
2-Chloro-5-[3-[(3-hydroxypropyl)amino]propyl]-N-(tricyclo([3.3.1.1]dec-1-ylmethyl)-benzamide hydrochloride,
2-Chloro-5-(piperidin-4-ylsulfinyl)-N-(tricyclo[3.3.1.1^{3,7}]dec-1-ylmethyl)-benzamide,
N-(1-Adamantylmethyl)-5-chloro-2-{3-[(3-hydroxypropyl)amino]propyl}-isonicotinamide dihydrochloride,
N-(1-Adamantylmethyl)-2-chloro-5-(3-{[(1R)-2-hydroxy-1-methylethyl]amino}propyl)nicotinamide,
N-(1-Adamantylmethyl)-5-chloro-2-[3-(ethylamino)propyl]isonicotinamide,
N-(1-Adamantylmethyl)-5-chloro-2-{3-[(2-hydroxyethyl)amino]propyl}-isonicotinamide,
N-(1-Adamantylmethyl)-5-chloro-2-(3-{[(2S)-2-hydroxypropyl]amino}propyl)isonicotinamide,
   or a pharmaceutically acceptable salt or solvate of any one thereof.

Pharmaceutically acceptable salts include, where applicable, acid addition salts derived from pharmaceutically acceptable inorganic and organic acids such as a chloride, bromide, sulphate, phosphate, maleate, fumarate, tartrate, citrate, benzoate, 4-methoxybenzoate, 2- or 4-hydroxybenzoate, 4-chlorobenzoate, p-toluenesulphonate, methanesulphonate, ascorbate, acetate, succinate, lactate, glutarate, gluconate, tricarballylate, hydroxynaphthalene-carboxylate or oleate salt; and salts prepared from pharmaceutically acceptable inorganic and organic bases. Salts derived from inorganic bases include aluminium, ammonium, calcium, copper, ferric, ferrous, lithium, magnesium, manganic, manganous, potassium, sodium, zinc and bismuth salts. Particularly preferred are the ammonium, calcium, magnesium, potassium and sodium salts. Salts derived from pharmaceutically acceptable organic bases include salts of primary, secondary and tertiary amines, cyclic amines like arginine, betaine, choline and the like.

Examples of pharmaceutically acceptable solvates include hydrates.

Examples of inhibitors of TACE include the compounds described in WO 99/18074, WO 99/65867, U.S. Pat. No. 6,225,311, WO 00/00465, WO 00/09485, WO 98/38179, WO 02/18326 and WO 02/096426, the entire contents of which are incorporated herein by reference.

In an embodiment of the invention, the TACE inhibitor is
3-Amino-N-hydroxy-α-(2-methylpropyl)-3-[4-[(2-methyl-4-quinolinyl)methoxy]phenyl]-2-oxo-1-pyrrolidineacetamide (also known as DPC-333),
2(S),3(S)-Piperidinedicarboxamide, N3-hydroxy-1-methyl-N-2-[4-[(2-methyl-4-quinolinyl)methoxy]phenyl],
3-Thiomorpholinecarboxamide, 4-[[4-(2-butynyloxy)phenyl]sulfonyl]-N-hydroxy-2, dimethyl (also known as TMI-1),
5-Hexenoic acid, 3-[(hydroxyamino)carbonyl]-2-(2-methylpropyl)-6-phenyl-, 2-(2-methylpropyl)-2-(methylsulfonyl)hydrazide, (2R,3S,5E) (also known as Ro 32-7315),
2-Piperidinecarboxamide, N,5-dihydroxy-1-[[4-(1-naphthalenylmethoxy)phenyl]sulfonyl]-, (2R,5R),
Pentanamide, 3-(formylhydroxyamino)-4-methyl-2-(2-methylpropyl)-N-[(1S,2S)-2-methyl-1-[(2-pyridinylamino)carbonyl]butyl]-, (2R,3S) (also known as GW 3333),
2-Propenamide, N-hydroxy-3-[3-[[(4-methoxyphenyl)sulfonyl](1-methylethyl)amino]phenyl]-3-(3-pyridinyl)-, (2E) (also known as W-3646),
Benzamide, N-(2,4-dioxo-1,3,7-triazaspiro[4.4]non-9-yl)-4-[(2-methyl-4-quinolinyl)methoxy],
Benzamide, N-[(1-acetyl-4-piperidinyl)(2,5-dioxo-4-imidazolidinyl)methyl]-4-[(2-methyl-4-quinolinyl)methoxy], or
2,4-Imidazolidinedione, 5-methyl-5-[[[4-[(2-methyl-4-quinolinyl)methoxy]phenyl]sulfonyl]methyl].

The invention also provides a pharmaceutical product comprising, in combination, a preparation of a first active ingredient which is a P2X₇ receptor antagonist, and a preparation of a second active ingredient which is an inhibitor of proTNFα convertase enzyme (TACE), for simultaneous, sequential or separate use in therapy.

In another aspect, the invention provides a kit comprising a preparation of a first active ingredient which is a P2X₇ receptor antagonist, a preparation of a second active ingredient which is an inhibitor of proTNFα convertase enzyme (TACE), and instructions for the simultaneous, sequential or separate administration of the preparations to a patient in need thereof.

It has been found that the choice of active ingredients according to the invention is advantageous because it results in a beneficial anti-inflammatory effect and, accordingly, can be used to treat various acute and chronic inflammatory conditions/disorders such as rheumatoid arthritis.

The pharmaceutical composition of the invention may be prepared by mixing the first active ingredient with the second active ingredient. Therefore, in a further aspect of the present invention, there is provided a process for the preparation of a pharmaceutical composition which comprises mixing a first active ingredient which is a P2X₇ receptor antagonist, with a second active ingredient which is an inhibitor of proTNFα convertase enzyme (TACE).

The first and second active ingredients may alternatively be administered simultaneously (other than in admixture as described above), sequentially or separately to treat inflammatory conditions. By sequential is meant that the first and second active ingredients are administered, in any order, one immediately after the other. They still have the desired effect if they are administered separately but less than about 4 hours apart, preferably less than about 2 hours apart, more preferably less than about 30 minutes apart.

The first and second active ingredients are conveniently administered by oral or parenteral administration using conventional systemic dosage forms, such as tablets, capsules, pills, powders, aqueous or oily solutions or suspensions, emulsions and sterile injectable aqueous or oily solutions or suspensions. These dosage forms will usually include one or more pharmaceutically acceptable ingredients which may be selected, for example, from adjuvants, carriers, binders, lubricants, diluents, stabilising agents, buffering agents, emulsifying agents, viscosity-regulating agents, surfactants, preservatives, flavourings and colorants.

Oral administration is preferred.

For the above-mentioned therapeutic uses the dosages administered will, of course, vary with the first and second active ingredients employed, the mode of administration, the treatment desired and the condition or disorder indicated. However, in general, satisfactory results will be obtained when the total, combined, daily dosage of first and second active ingredients, when taken orally, is in the range from 10 to 500 milligrammes (mg), particularly from 10, 20, 30, 40 or 50 to 450, preferably to 400, more preferably to 300 mg.

The pharmaceutical composition, pharmaceutical product or kit according to the invention may be administered as divided doses from 1 to 4 times a day, and preferably once or twice a day.

The present invention further provides the use of a pharmaceutical composition, pharmaceutical product or kit according to the invention in the manufacture of a medicament for the treatment of an inflammatory disorder.

Also, the present invention provides a method of treating an inflammatory disorder which comprises administering a therapeutically effective amount of a pharmaceutical composition of the invention to a patient in need thereof.

Still further, the present invention provides a method of treating an inflammatory disorder which comprises simultaneously, sequentially or separately administering:

(a) a (therapeutically effective) dose of a first active ingredient which is a P2X₇ receptor antagonist; and

(b) a (therapeutically effective) dose of a second active ingredient which is an inhibitor of proTNFα convertase enzyme (TACE),

to a patient in need thereof.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the condition or disorder in question. Persons at risk of developing a particular condition or disorder generally include those having a family history of the condition or disorder, or those who have been identified by genetic testing or screening to be particularly susceptible to developing the condition or disorder.

The present invention will now be further understood by reference to the following illustrative examples.

### EXAMPLE 1

Pharmacological Analysis to Determine the Effect of TACE Inhibitor/P2X₇ Antagonist Combinations (without Addition of a P2X₇ Agonist).

Human peripheral blood from healthy human volunteers was collected in lithium-heparin blood tubes. Test mixtures were added and the blood was incubated at 37 degrees centigrade for 15-60 minutes. Test mixtures can compromise of vehicle as control, a P2X₇ receptor antagonist, or a combination of a P2X₇ receptor antagonist together with a TACE inhibitor. Lipopolysacharide (LPS) was then added to the blood and this was incubated for a further 3-6 hours at 37 degrees centigrade. After incubation, samples of cell supernatants were transferred to a 96-well plate for subsequent cytokine and mediator measurements. The formation of inflammatory mediators was measured in the cell supernatant, by specific ELISA for cytokines, including IL-1, IL-18, TNFα, IL2, IL6, IL8, and for other mediators including PGE2, NO and matrix metalloproteinases (MMPs). The levels of mediators released in the presence of a P2X₇ receptor antagonist alone, or in the presence of a TACE inhibitor alone, or in the presence of a combination of a P2X₇ receptor antagonist with a TACE inhibitor were determined. The effects of the antagonists/inhibitors alone and in combination were then compared. Statistically significant levels of inhibitory activity against a single mediator or on multiple mediators by P2X₇ antagonist/TACE inhibitor combinations, in comparison to that achieved by either a P2X₇ antagonist or a TACE inhibitor alone, is an indicator for increased efficacy in the treatment of disease.

### EXAMPLE 2

Pharmacological Analysis to Determine the Effect of TACE Inhibitor/P2X₇ Anatagonist Combinations (with Addition of a P2X₇ Agonist).

Human peripheral blood from healthy human volunteers was collected in lithium-heparin blood tubes. Test mixtures were added to the blood and incubated at 37 degrees centrigrade for 15-60 minutes. Test mixtures can compromise of vehicle as control, a P2X₇ receptor antagonist, or a combination of a P2X₇ receptor antagonist together with a TACE inhibitor. Lipopolysacharide (LPS) was then added to the blood and this was incubated for a further 3-6 hours at 37 degrees centigrade. The P2X₇ receptor agonist ATP was added and after incubation for a further 30 minutes at 37 degrees centigrade, samples of blood supernatants were transferred to a 96-well plate for subsequent cytokine and mediator measurements. The formation of inflammatory mediators was measured in the cell supernatant, by specific ELISA for cytokines, including IL-1, IL-18, TNFα, IL2, IL6, IL8, and for other mediators including PGE2, NO and matrix metalloproteinases (MMPs). The levels of mediators released in the presence of a P2X₇ receptor antagonist alone, or in the presence of a combination of a P2X₇ receptor antagonist with a TACE inhibitor were determined. The effects produced by a P2X₇ antagonist alone and in combination with a TACE inhibitor were then compared. Statistically significant levels of inhibitory activity against a single mediator or on multiple mediators by P2X₇ antagonist/TACE inhibitor combinations in comparison to that achieved by a P2X₇ antagonist alone is an indicator for increased efficacy in the treatment of disease.

1. A pharmaceutical composition comprising, in admixture, a first active ingredient which is a P2X₇ receptor antagonist, and a second active ingredient which is an inhibitor of proTNFα convertase enzyme (TACE).

2. A composition according to claim 1, wherein the P2X₇ receptor antagonist is an adamantyl derivative.

3. A composition according to claim 1 or claim 2, wherein the P2X₇ receptor antagonist is a compound of formula 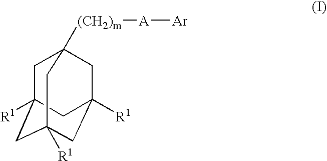
wherein m represents 1, 2 or 3;
each R¹ independently represents a hydrogen or halogen atom;
A represents C(O)NH or NHC(O);
Ar represents a group 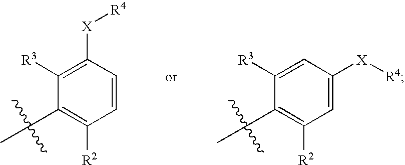
X represents a bond, an oxygen atom or a group CO, (CH₂)₁₋₆, CH═, (CH₂)₁₋₆O, O(CH₂)₁₋₆, O(CH₂)₂₋₆O, O(CH₂)₂₋₃O(CH₂)₁₋₃, CR′(OH), (CH₂)₁₋₃O(CH₂)₁₋₃, (CH₂)₁₋₃O(CH₂)₂₋₃O, NR⁵, (CH₂)₁₋₆NR⁵, NR⁵(CH₂)₁₋₆, (CH₂)₁₋₃NR⁵(CH₂)₁₋₃, O(CH₂)₂₋₆NR⁵, O(CH₂)₂₋₃NR⁵(CH₂)₁₋₃, (CH₂)₁₋₃NR⁵(CH₂)₂₋₃O, NR⁵(CH₂)₂₋₆O, NR⁵(CH₂)₂₋₃O(CH₂)₁₋₃, CONR⁵, NR⁵CO, S(O)ₙ, S(O)ₙCH₂, CH₂S(O)ₙ, SO₂NR⁵ or NR⁵SO₂;
n is 0, 1 or 2;
R′ represents a hydrogen atom or a C₁-C₆ alkyl group;
one of R² and R³ represents a halogen, cyano, nitro, amino, hydroxyl, or a group selected from (i) C₁-C₆ alkyl optionally substituted by at least one C₃-C₆ cycloalkyl, (ii) C₃-C₈ cycloalkyl, (iii) C₁-C₆ alkyloxy optionally substituted by at least one C₃-C₆ cycloalkyl, and (iv) C₃-C₈ cycloalkyloxy, each of these groups being optionally substituted by one or more fluorine atoms, and the other of R² and R³ represents a hydrogen or halogen atom;
either R⁴ represents a 3- to 9-membered saturated or unsaturated aliphatic heterocyclic ring system containing one or two nitrogen atoms and optionally an oxygen atom, the heterocyclic ring system being optionally substituted by one or more substituents independently selected from fluorine atoms, hydroxyl, carboxyl, cyano, C₁-C₆ alkyl, C₁-C₆ hydroxyalkyl, —NR⁶R⁷, —(CH₂)ᵣNR⁶R⁷ and —CONR⁶R⁷,
or R⁴ represents a 3- to 8-membered saturated carbocyclic ring system substituted by one or more substituents independently selected from —NR⁶R⁷, —(CH₂)ᵣNR⁶R⁷ and —CONR⁶R⁷, the ring system being optionally further substituted by one or more substituents independently selected from fluorine atoms, hydroxyl and C₁-C₆ alkyl;
r is 1, 2, 3, 4, 5 or 6;
R⁵ represents a hydrogen atom or a C₁-C₆ alkyl or C₃-C₈ cycloalkyl group;
R⁶ and R⁷ each independently represent a hydrogen atom or a C₁-C₆ alkyl, C₂-C₆ hydroxyalkyl or C₃-C₈ cycloalkyl group, or R⁶ and R⁷ together with the nitrogen atom to which they are attached form a 3- to 8-membered saturated heterocyclic ring;
with the provisos that,
(a) when A represents C(O)NH and R⁴ represents an unsubstituted 3- to 8-membered saturated aliphatic heterocyclic ring system containing one nitrogen atom, then X is other than a bond, and
(b) when A represents C(O)NH and X represents a group (CH₂)₁₋₆ or O(CH₂)₁₋₆, then R⁴ does not represent an unsubstituted imidazolyl, unsubstituted morpholinyl, unsubstituted piperidinyl or unsubstituted pyrrolidinyl group, and
(c) when A represents NHC(O) and R⁴ represents an unsubstituted 3- to 8-membered saturated aliphatic heterocyclic ring system containing one nitrogen atom, then X is other than a bond, and
(d) when A represents NHC(O) and X represents O(CH₂)₁₋₆, NH(CH₂)₁₋₆ or SCH₂, then R⁴ does not represent an unsubstituted 1-piperidinyl or unsubstituted 1-pyrrolidinyl group, and
(e) when A represents NHC(O) and X represents O(CH₂)₂₋₃NH(CH₂)₂, then R⁴ does not represent an imidazolyl group;
or a pharmaceutically acceptable salt or solvate thereof.

4. A composition according to claim 1, wherein the P2X₇ receptor antagonist is a compound of formula 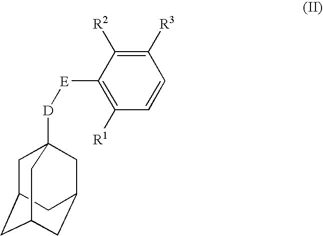
wherein D represents CH₂ or CH₂CH₂;
E represents C(O)NH or NHC(O);
R¹ and R² each independently represent a hydrogen or halogen atom, or an amino, nitro, C₁-C₆ alkyl or trifluoromethyl group;
R³ represents a group of formula 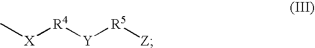
X represents an oxygen or sulphur atom or a group NH, SO or SO₂;
Y represents an oxygen or sulphur atom or a group NR¹¹, SO or SO₂;
Z represents a group —OH, —SH, —CO₂H, C₁-C₆ alkoxy, C₁-C₆ alkylthio, C₁-C₆-alkylsulphinyl, C₁-C₆-alkylsulphonyl, —NR⁶R⁷, —C(O)NR⁸R⁹, imidazolyl, 1-methylimidazolyl, —N(R¹⁰)C(O)—C₁-C₆ alkyl, C₁-C₆ alkylcarbonyloxy, C₁-C₆ alkoxycarbonyloxy, —OC(O)NR¹²R¹³, —OCH₂OC(O)R¹⁴, —OCH₂OC(O)OR¹⁵ or —OC(O)OCH₂OR¹⁶;
R⁴ represents a C₂-C₆ alkyl group;
R⁵ represents a C₁-C₆ alkyl group;
R⁶, R⁷, R⁸, R⁹, R¹⁰, R¹² and R¹³ each independently represent a hydrogen atom, or a C₁-C₆ alkyl group optionally substituted by at least one hydroxyl group;
R¹¹ represents a hydrogen atom, or a C₁-C₆ alkyl group optionally substituted by at least one substituent independently selected from hydroxyl and C₁-C₆ alkoxy; and
R¹⁴, R¹⁵ and R¹⁶ each independently represent a C₁-C₆ alkyl group;
with the provisos that (i) when E represents NHC(O), X represents O, S or NH and Y represents O, then Z represents —NR⁶R⁷ where R⁶ represents a hydrogen atom and R⁷ represents either a hydrogen atom or a C₁-C₆ alkyl group substituted by at least one hydroxyl group, and (ii) when E represents NHC(O), X represents O, S or NH, Y represents NH and R⁵ represents CH₂CH₂, then Z is not —OH or imidazolyl;
or a pharmaceutically acceptable salt or solvate thereof.

5. A composition according to claim 1, wherein the P2X₇ receptor antagonist is discloses a compound of formula 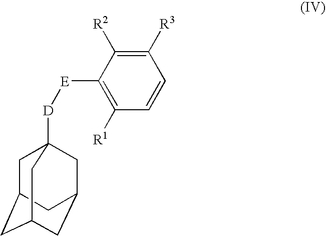
wherein D represents CH₂ or CH₂CH₂;
E represents C(O)NH or NHC(O);
R¹ and R² each independently represent hydrogen, halogen, amino, nitro, C₁-C₆ alkyl or trifluoromethyl, but R¹ and R² may not both simultaneously represent hydrogen;
R³ represents a group of formula 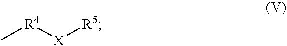
R⁴ represents a C₁-C₆ alkyl group;
X represents an oxygen or sulphur atom or a group NR¹³, SO or SO₂;
R⁵ represents hydrogen, or R⁵ represents C₁-C₆ alkyl or C₂-C₆ alkenyl, each of which may be optionally substituted by at least one substituent selected from halogen, hydroxyl, (di)-C₁-C₆-alkylamino, —Y—R⁶, 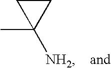
a 5- or 6-membered heteroaromatic ring comprising from 1 to 4 heteroatoms independently selected from nitrogen, oxygen and sulphur which heteroaromatic ring may itself be optionally substituted by at least one substituent selected from halogen, hydroxyl and C₁-C₆ alkyl;
Y represents an oxygen or sulphur atom or a group NH, SO or SO₂;
R⁶ represents a group —R⁷Z where R⁷ represents a C₂-C₆ alkyl group and Z represents an —OH, —CO₂H, —NR⁸R⁹, —C(O)NR¹⁰R¹¹ or —N(R¹²)C(O)—C₁-C₆ alkyl group, and,
in the case where Y represents an oxygen or sulphur atom or a group NH, R⁶ additionally represents hydrogen, C₁-C₆ alkyl, C₁-C₆ alkylcarbonyl, C₁-C₆ alkoxycarbonyl, —C(O)NR¹⁴R¹⁵, —CH₂OC(O)R¹⁶, —CH₂OC(O)OR¹⁷ or —C(O)OCH₂OR¹⁸;
R⁸, R⁹, R¹⁰, R¹¹ and R¹² each independently represent a hydrogen atom or a C₁-C₆ alkyl group;
R¹³ represents hydrogen, C₃-C₈ cycloalkyl, C₃-C₈ cycloalkylmethyl, or R¹³ represents a C₁-C₆ alkyl group optionally substituted by at least one substituent selected from hydroxyl and C₁-C₆ alkoxy; and
R¹⁴, R¹⁵, R¹⁶, R¹⁷ and R¹⁸ each independently represent a C₁-C₆ alkyl group;
with the proviso that when E is C(O)NH, X is O, NH or N(C₁-C₆ alkyl), then R⁵ is other than a hydrogen atom or an unsubstituted C₁-C₆ alkyl group;
or a pharmaceutically acceptable salt or solvate thereof.

6. A composition according to claim 1, wherein the P2X₇ receptor antagonist is a compound of formula 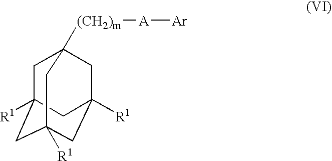
wherein m represents 1, 2 or 3;
each R¹ independently represents a hydrogen or halogen atom;
A represents C(O)NH or NHC(O);
Ar represents a group 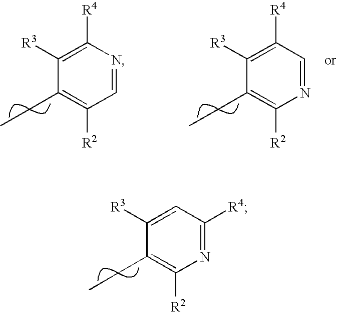
one of R² and R³ represents halogen, nitro, amino, hydroxyl, or a group selected from (i) C₁-C₆ alkyl optionally substituted by at least one halogen atom, (ii) C₃-C₈ cycloalkyl, (iii) C₁-C₆ alkoxy optionally substituted by at least one halogen atom, and (iv) C₃-C₈ cycloalkyloxy, and the other of R² and R³ represents a hydrogen or halogen atom;
R⁴ represents a group 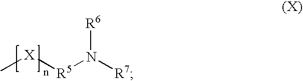
X represents an oxygen or sulphur atom or a group >N—R⁸;
n is 0 or 1;
R⁵ represents a C₁-C₅ alkyl group which may be optionally substituted by at least one substituent selected from hydroxyl, halogen and C₁-C₆ alkoxy;
R⁶ and R⁷ each independently represent a hydrogen atom, C₁-C₆ alkyl (optionally substituted by at least one substituent selected from hydroxyl, halogen, C₁-C₆ alkoxy, and (di)-C₁-C₄ alkylamino (itself optionally substituted by at least one hydroxyl group)), or C₃-C₈ cycloalkyl (optionally substituted by at least one substituent selected from hydroxyl, halogen and C₁-C₆ alkoxy); and
R⁸ represents a hydrogen atom or a C₁-C₅ alkyl group which may be optionally substituted by at least one substituent selected from hydroxyl, halogen and C₁-C₆ alkoxy;
with the provisos that:
(a) when n is 0, then A is NHC(O), and
(b) when n is 1, X represents oxygen and A is C(O)NH, then R⁶ and R⁷ do not both simultaneously represent a hydrogen atom or do not both simultaneously represent an unsubstituted C₁-C₆ alkyl, or when one of R⁶ and R⁷ represents a hydrogen atom, then the other of R⁶ and R⁷ does not represent an unsubstituted C₁-C₆ alkyl; and
(c) when n is 1, X is oxygen, sulphur or >NH and A is NHC(O), then R⁶ and R⁷ do not both simultaneously represent a hydrogen atom or do not both simultaneously represent an unsubstituted C₁-C₆ alkyl, or when one of R⁶ and R⁷ represents a hydrogen atom, then the other of R⁶ and R⁷ does not represent an unsubstituted C₁-C₆ alkyl or —CH₂CH₂OH;
or a pharmaceutically acceptable salt or solvate thereof.

7. A composition according to claim 1, wherein the P2X₇ receptor antagonist is:
2-Chloro-5-[[2-(2-hydroxy-ethylamino)-ethylamino]-methyl]-N-(tricyclo[3.3.1.1^{3,7}]dec-1-ylmethyl)-benzamide, dihydrochloride,
2-Chloro-5-[3-[(3-hydroxypropyl)amino]propyl]-N-(tricyclo[3.3.1.1]dec-1-ylmethyl)-benzamide,
(R)-2-Chloro-5-[3-[(2-hydroxy-1-methylethyl)amino]propyl]-N-(tricyclo[3.3.1.1^{3,7}]dec-1-ylmethyl)-benzamide,
2-Chloro-5-[[2-[(2-hydroxyethyl)amino]ethoxy]methyl]-N-(tricyclo[3.3.1.1^{3,7}]dec-1-ylmethyl)-benzamide,
2-Chloro-5-[3-[3-(methylamino)propoxy]propyl]-N-(tricyclo[3.3.1.1^{3,7}]dec-1-ylmethyl)benzamide,
2-Chloro-5-[3-(3-hydroxy-propylamino)-propoxy]-N-(tricyclo[3.3.1.1^{3,7}]dec-1-ylmethyl)-benzamide,
2-Chloro-5-[2-(3-hydroxypropylamino)ethylamino]-N-(tricyclo[3.3.1.1^{3,7}]dec-1-ylmethyl)-benzamide,
2-Chloro-5-[2-(3-hydroxypropylsulfonyl)ethoxy]-N-(tricyclo[3.3.1.1^{3,7}]dec-1-ylmethyl)-benzamide,
2-Chloro-5-[2-[2-[(2-hydroxyethyl)amino]ethoxy]ethoxy]-N-(tricyclo[3.3.1.1^{3,7}]dec-1-ylmethyl)-benzamide,
2-Chloro-5-[[2-[[2-(1-methyl-1H-imidazol-4-yl)ethyl]amino]ethyl]amino]-N-(tricyclo[3.3.1.1^{3,7}]dec-1-ylmethyl)-benzamide,
2-Chloro-5-piperazin-1-ylmethyl-N-(tricyclo[3.3.1.1]dec-1-ylmethyl)-benzamide,
2-Chloro-5-(4-piperidinyloxy)-N-(tricyclo[3.3.1.1^{3,7}]dec-1-ylmethyl)-benzamide,
2-Chloro-5-(2,5-diazabicyclo[2.2.1]hept-2-ylmethyl)-N-(tricyclo[3.3.1.1]dec-1-ylmethyl)-benzamide,
2-Chloro-5-[3-[(3-hydroxypropyl)amino]propyl]-N-(tricyclo[3.3.1.1]dec-1-ylmethyl)-benzamide hydrochloride,
2-Chloro-5-(piperidin-4-ylsulfinyl)-N-(tricyclo[3.3.1.1^{3,7}]dec-1-ylmethyl)-benzamide,
N-(1-Adamantylmethyl)-5-chloro-2-{3-[(3-hydroxypropyl)amino]propyl}-isonicotinamide dihydrochloride,
N-(1-Adamantylmethyl)-2-chloro-5-(3-{[(1R)-2-hydroxy-1-methylethyl]amino}propyl)nicotinamide,
N-(1-Adamantylmethyl)-5-chloro-2-[3-(ethylamino)propyl]isonicotinamide,
N-(1-Adamantylmethyl)-5-chloro-2-{3-[(2-hydroxyethyl)amino]propyl}-isonicotinamide,
N-(1-Adamantylmethyl)-5-chloro-2-(3-{[(2S)-2-hydroxypropyl]amino}propyl)isonicotinamide,
or a pharmaceutically acceptable salt or solvate of any one thereof.

8. A composition according to claim 1, wherein the inhibitor of proTNFα convertase enzyme is:
3-Amino-N-hydroxy-α-(2-methylpropyl)-3-[4-[(2-methyl-4-quinolinyl)methoxy]phenyl]-2-oxo-1-pyrrolidineacetamide,
2(S),3(S)-Piperidinedicarboxamide, N3-hydroxy-1-methyl-N-2-[4-[(2-methyl-4-quinolinyl)methoxy]phenyl],
3-Thiomorpholinecarboxamide, 4-[[4-(2-butynyloxy)phenyl]sulfonyl]-N-hydroxy-2,2-dimethyl,
5-Hexenoic acid, 3-[(hydroxyamino)carbonyl]-2-(2-methylpropyl)-6-phenyl-, 2-(2-methylpropyl)-2-(methylsulfonyl)hydrazide, (2R,3S,5E),
2-Piperidinecarboxamide, N,5-dihydroxy-1-[[4-(1-naphthalenylmethoxy)phenyl]sulfonyl]-, (2R,5R),
Pentanamide, 3-(formylhydroxyamino)-4-methyl-2-(2-methylpropyl)-N-[(1S,2S)-2-methyl-1-[(2-pyridinylamino)carbonyl]butyl]-, (2R,3S),
2-Propenamide, N-hydroxy-3-[3-[[(4-methoxyphenyl)sulfonyl](1-methylethyl)amino]phenyl]-3-(3-pyridinyl)-, (2E),
Benzamide, N-(2,4-dioxo-1,3,7-triazaspiro[4.4]non-9-yl)-4-[(2-methyl-4-quinolinyl)methoxy],
Benzamide, N-[(1-acetyl-4-piperidinyl)(2,5-dioxo-4-imidazolidinyl)methyl]-4-[(2-methyl-4-quinolinyl)methoxy], or
2,4-Imidazolidinedione, 5-methyl-5-[[[4-[(2-methyl-4-quinolinyl)methoxy]phenyl]sulfonyl]methyl].

9. A composition according to claim 1 which is formulated for oral administration.

10. A process for the preparation of a pharmaceutical composition as defined in claim 1 which comprises mixing the first active ingredient with the second active ingredient.

11-12. (canceled)

13. A method of treating an inflammatory disorder which comprises administering a therapeutically effective amount of a pharmaceutical composition as defined in claim 1 to a patient in need thereof.

14. A method according to claim 13, wherein the inflammatory disorder is rheumatoid arthritis.

15. A pharmaceutical product comprising, in combination, a preparation of a first active ingredient which is a P2X₇ receptor antagonist, and a preparation of a second active ingredient which is an inhibitor of proTNFα convertase enzyme (TACE), for simultaneous, sequential or separate use in therapy.

16. A kit comprising a preparation of a first active ingredient which is a P2X₇ receptor antagonist, a preparation of a second active ingredient which is an inhibitor of proTNFα convertase enzyme (TACE), and instructions for the simultaneous, sequential or separate administration of the preparations to a patient in need thereof.
